SEQUENCE LISTING (6 sequences)

SEQ ID NO: 1 - DNA, 20 bp, Artificial
  primer synthesis
accactcgcattcctttgac

SEQ ID NO: 2 - DNA, 20 bp, Artificial
  primer synthesis
tcagcgggaaggactttatg

SEQ ID NO: 3 - DNA, 20 bp, Artificial
  primer synthesis
aggtgctggagttgaccagt

SEQ ID NO: 4 - DNA, 20 bp, Artificial
  primer synthesis
cagcctagagatccagcgac

SEQ ID NO: 5 - DNA, 20 bp, Artificial
  primer synthesis
gtgttcctacccccaatgtg

SEQ ID NO: 6 - DNA, 20 bp, Artificial
  primer synthesis
cttgctcagtgtccttgctg

(19) United States
(12) Patent Application Publication
(10) Pub. No.: US 2011/0124742 A1
(43) Pub. Date: May 26, 2011

(54) COMPOSITION AND METHOD OF ADIPOSE CELL DIFFERENTIATION INHIBITION

(75) Inventors: MENG-HWAN LEE, TAIPEI CITY (TW); JUNG-WEI TSAI, TAIPEI CITY (TW); YUN-YU CHEN, TAIPEI CITY (TW); YING-CHIEH TSAI, TAIPEI CITY (TW); CHUN-YING LIN, TAIPEI CITY (TW)

(73) Assignees: NATIONAL YANG-MING UNIVERSITY, Taipei City (TW); YANGSON BIOTECHNOLOGY CO., LTD., Taipei City (TW)

(21) Appl. No.: 13/018,163

(22) Filed: Jan. 31, 2011

Related U.S. Application Data
(62) Division of application No. 12/193,359, filed on Aug. 18, 2008.

(51) Int. Cl. A61K 31/09; A61P 19/10; A61P 3/04; A61P 3/00

(52) U.S. Cl. 514/720

(57) ABSTRACT

The present invention is related to a composition and method of adipose cell differentiation inhibition.

### CROSS-REFERENCE TO RELATED APPLICATION

This application is a Divisional of the pending U.S. patent application Ser. No. 12/193,359 filed on Aug. 18, 2008, and is hereby incorporated by reference in its entirety.

Although incorporated by reference in its entirety, no arguments or disclaimers made in the parent application apply to this divisional application. Any disclaimer that may have occurred during the prosecution of the above-referenced application(s) is hereby expressly rescinded. Consequently, the Patent Office is asked to review the new set of claims in view of the entire prior art of record and any search that the Office deems appropriate.

### FIELD OF THE INVENTION

The present invention is related to a composition and method of adipose cell differentiation inhibition.

### BACKGROUND OF THE INVENTION

In the past several decades, change of people's eating habit and life style is related to steady increase of human's average weight. The overweight issue is not only a physiological burden but also a health problem. For a healthy person, extra energy will be transformed into lipid in adipose cells, and other types of cells can only maintain least amount of triglyceride. However, for an overweight person, adipose tissue can not only store extra amount of lipid, but lead to abnormal lipid accumulation in other tissues(Flier, J. S., Obesity wars: molecular progress confronts an expanding epidemic. Cell 2004, 116, 337-50.). From previous study, over-accumulated triglyceride in muscle, liver and spleen cells can cause physiological malfunction as well as inducing type II diabetes, high blood pressure, heart disease, arthritis or even cancer. It became a great concern to health(Camp, H. S.; Ren, D.; Leff, T., Adipogenesis and fat-cell function in obesity and diabetes. Trends Mol. Med. 2002, 8, 442-47; Flier, J. S., Obesity wars: molecular progress confronts an expanding epidemic. Cell 2004, 116, 337-50; Gimeno, R. E.; Klaman, L. D., Adipose tissue as an active Endocrine organ: recent advances. Curr. Opin. Pharmacol. 2005, 5, 122-28).

Adipose tissue includes white adipose tissue (WAT) and brown adipose tissue (BAT) which content and distribution varies depending on species. White adipose tissue is the major energy storage site for higher eukaryotes. When extra cellular energy turns into triglyceride, it will be stored here. On the other hand, when cell is lacking energy, triglyceride stored in white adipose tissue can be transformed into energy to release (Ntambi, J. M.; Kim, Y. C., Adipocyte Differentiation and Gene Expression. J. Nutr. 2000, 130, 3122S-33126). Therefore, the development level of white adipose tissue is a major factor of regulating inner energy homeostasis. The major cause of overweight is due to white adipose tissue increase.

In general, adipose cell can be grouped into several different stages. The first stage is growth arrest; adipose precursor cell must leave cell cycle to arrest before differentiation usually through cell-cell contact inhibition. The second stage is clonal expansion; if cell receives differentiation signal, it will go through at least once chromosomal replication and cell division. In order to adopt later differentiation process, genes responsible for regulating cell shape changes need to be turned on. Unwinding chromosome during cell division process is believed to be helpful for transcription factor getting into the regulatory region of these genes. At this stage, cell is transformed gradually from spindle shape into cytoplasmic extended shape (Gregoire, F. M., Adipocyte differentiation: from fibroblast to endocrine cell. Exp. Biol. Med. 2001, 226, 997-1002; Gregoire, F. M.; Smas, C. M.; Sul, H. S., Understanding adipocyte differentiation. Physiol. Rev. 1998, 78, 783-809; Ntambi, J. M.; Kim, Y. C., Adipocyte Differentiation and Gene Expression. J. Nutr. 2000, 130, 3122S-33126; Feve, B., Adipogenesis: cellular and molecular aspects. Best Pract. Res. Clin. Endocrinol. Metab. 2005, 19, 483-99). The last stage is terminal differentiation; after 5-7 days of differentiation, adipose cell is shown as mature round adipose cell morphology, and triglyceride oil drop is accumulated inside the cell. The marker gene specific to terminal differentiation is starting to express. Activated protein or mRNA includes triglyceride metabolism related enzyme, carbohydrate transported protein, insulin receptor and some adipose secretary hormone secreting into blood. Usually, after 8-12 days of differentiation can generate mature adipose cells.

To summarize, adipose cell differentiation can be treated as transcription factor remodeling which leads to a series of adipose tissue related gene expression. Current research discovered that three families of transcription factors are involved in lipid biosynthesis process. The first is C/EBPα, -β, -δ, the second is PPARγ, and the third is ADD1.

Plant extract contains complex chemical composition. Utilizing the components with aromatic smell and physiological activity to improve health is recognized as one specialized knowledge, aromatherapy. Aromatherapy is an integrated therapy, which is art and science using plant aromatic essence to regulate physiology, psychology and spirit. So called aromatic essence usually means essential oil of plant which is extracted by distillation, steaming and boiling, or solvent extraction from natural plants. Current aromatherapy is using essential oil extracted from various parts of plants to improve human being's physiological and psychological health, improve life quality or add pleasure (Thomas, D. V., Aromatherapy: mythical, magical, or medicinal? Holist. Nurs. Pract. 2002, 16, 8-16). Some essential oil products are claimed for their weight loosing function, and most of them are lacking scientific evidence. The composition of essential oil is very complicated. One essential oil may contain dozens to hundreds of components, and most of them are monoterpenes/C10 and sesquiterpenes/C15 compounds which exhibit stronger aroma and diversified physiological activity and usually used as major ingredient for medical, food, and cosmetics.

Acorus sp. is a perennial herb which belongs to Araceae family. The rhizome grows horizontally on the ground with brown yellow skin and fleshy root with fibrous root system on it. Upper rhizome has lots of branches; therefore, the paint is in a bush-like form. It grows in Europe, west Asia and North America. This plant prefers cold and humid climate and damp area. It exhibits cold but not drout resistance. In India, essential oil extracted from Acorus is used as pesticide due to the anti-insect physiological activity of Indian Acorus rhizome. In Ayurvedic medicine, Acorus rhizome can be used for an anticonvulsant agent, cold relief, gastroenterological parasite exterminator as well as mental illness treatment such as epilepsy. Essential oil extracted from rhizome of Europe derived Acorus calamus is used mostly in fragrance industry. The different application of Acorus essential oil results from the composition variation of essential oil extracted from Indian and European species (Marongiu, B.; Piras, A.; Porcedda, S.; Scorciapino, A., Chemical composition of the essential oil and supercritical CO2 extract of Commiphora myrrha (Nees) Engl. and of Acorus calamus L. J. Agric. Food Chem. 2005, 53, 7939-43). Local Chinese Acorus tatarinowii Schott grows at wetland under forest or on stones by river side over the altitude of 20˜2600 meters. It is distributed at south of yellow river basin. The major use is to treat fever, apoplexy, short memory, tinnitus, deaf, bloat, and muscle and joint pains associated with arthritis, strains, bruises, and sprains and etc. Related pharmacological study of Acorus includes its effect on central nervous system, its effect against cardiac arrhythmia, and its antibacterial and pesticidal activity.

### SUMMARY OF THE INVENTION

The present invention is related to a composition and method of adipose cell differentiation inhibition.

### BRIEF DESCRIPTION OF THE DRAWINGS

FIG. 1 shows morphology of 3T3-L1 adipose cell. (a) Undifferentiated 3T3-L1 pre-adipose cell is shown as spindle-shaped fibroblast mother cell. (b) Completely differentiated (day 9) mature adipose cell is shown as round-shaped with oil drop accumulation in the cell.

FIG. 2 shows the effect of Acorus essential oil on 3T3-L1 adipose cell. (a) Acorus inhibition effect on adipose cell differentiation. With increasing concentration of Acorus essential oil (0, 25, 62.5, and 125 μg/ml), triglyceride content in adipose cell is relatively decreasing at day 9. Normal differentiation control is added 0.1% DMSO. (b) Analysis of various concentrations of Acorus essential oil on adipose cell viability. Normal differentiation control is added 0.1% DMSO. Result is mean±standard deviation from three independent experiments. * represents student t-test analysis with significant difference relative to normal differentiation control, p<0.01.

FIG. 3 shows oil red staining of Acorus essential oil inhibition on 3T3-L1 adipose cell. (a) is normal differentiation control with 0.1% DMSO addition. (b)˜(d) represent 3T3-L1 adipose cell treated with 25, 62.5, and 125 μg/ml of Acorus essential oil. (e) shows 3T3-L1 adipose cell without differentiation solution addition. Staining is performed at day 9 of complete differentiation.

FIG. 4 is a gas chromatogram of Acorus essential oil.

FIG. 5 shows analysis of adipose cell differentiation inhibition with Acorus essential oil fractions.

FIG. 6 shows a HPLC chromatogram of Acorus essential oil second fraction.

FIG. 7 show α-asarone effect on 3T3-L1 adipose cell. (a) Inhibition effect of α-asarone at various concentrations (0, 0.015, 0.03, 0.06, 0.13, and 0.25 mM) on 3T3-L1 adipose cells. Normal differentiation control is added 0.1% DMSO. (b) Analysis of α-asarone effect on adipose cell viability. Normal differentiation control is added 0.1% DMSO. Result is mean±standard deviation from three independent experiments. * represents student t-test analysis with significant difference relative to normal differentiation control, p<0.01.

FIG. 8 shows β-asarone effect on 3T3-L1 adipose cell. (a) Inhibition effect of β-asarone at various concentrations (0, 0.015, 0.03, 0.06, 0.13, and 0.25 mM) on 3T3-L1 adipose cells. Normal differentiation control is added 0.1% DMSO. (b) Analysis of β-asarone effect on adipose cell viability. Normal differentiation control is added 0.1% DMSO. Result is mean±standard deviation from three independent experiments. * represents student t-test analysis with significant difference relative to normal differentiation control, p<0.01.

FIG. 9 shows oil red staining of β-asarone inhibition effect at various concentrations on 3T3-L1 adipose cell. (a) Normal differentiation control is added 0.1% DMSO. (b)˜(e) represent 3T3-L1 adipose cell treated with 0.03, 0.06, 0.13, and 0.25 mM of β-asarone. (f) shows 3T3-L1 adipose cell without differentiation solution addition. Staining is performed at day 9 of complete differentiation.

FIG. 10 shows β-asarone effect on PPARγ mRNA expression. When using various amounts of β-asarone to treat 3T3-L1 adipose cells, 0.25 mM of β-asarone treated sample shows significant decrease on PPARγ expression. Result is shown as mean±standard deviation from three independent experiments. * represents student t-test analysis with significant difference relative to normal differentiation control, p<0.01. GAPDH is used as an internal control.

FIG. 11 shows β-asarone effect on C/EBPα mRNA expression. When using various amounts of β-asarone to treat 3T3-L1 adipose cells, 0.25 mM of β-asarone treated sample shows significant decrease on C/EBPα expression. Result is shown as mean±standard deviation from three independent experiments. * represents student t-test analysis with significant difference relative to normal differentiation control, p<0.01. GAPDH is used as an internal control.

FIG. 12 shows β-asarone effect on PPARγ protein expression. When using various amounts of β-asarone to treat 3T3-L1 adipose cells, 0.25 mM of β-asarone treated sample shows significant decrease of PPARγ expression. α-tubulin is used as an internal control.

FIG. 13 shows β-asarone effect on C/EBPα protein expression. When using various amounts of β-asarone to treat 3T3-L1 adipose cells, 0.13 mM of β-asarone treated sample shows significant decrease of C/EBPα expression. α-tubulin is used as an internal control.

### DETAILED DESCRIPTION OF THE INVENTION

Essential oil extracted from herbal plants may very likely contain active components for overweight prevention. The present invention develops active components from herbal essential oil for effective overweight treatment. Taking 3T3-L1 adipose precursor cell line as a cell model and triglyceride content as an evaluation standard for cell differentiation, Acorus essential oil is identified by its effect on adipose cell differentiation inhibition. Further purification of Acorus essential oil identifies its active component, and we examine the effect of this active component on adipose cell differentiation inhibition. Demonstration of its effect and mechanism on adipose cell in the present invention is hopeful for overweight treatment in the future.

The essential oil extracted from Acorus rhizome contains α-, β-, and γ-asarone, euasarone, cis-methylisoeugenol, elemicin, asarylaldehyde, δ-cadinene, thymol and myristic acid. β-asarone is the major component in Acorus essential oil.

The present invention is the first invention discovering that Acorus essential oil can reduce triglyceride content and inhibit adipose cell differentiation. Further analysis demonstrates that its activity is from β-asarone. Effective amount of β-asarone can inhibit mRNA and protein expression of adipose cell transcription factor PPARγ and C/EBPα. Besides, lack of PPARγ can promote osteoblastogenesis and increase in vivo bone mass. Therefore, the present invention can also treat osteoporosis (Liming Pei and Peter Tontonoz, 2004, Fat's Loss is Bone's Gain, The Journal of Clinical Investigation, 113(6): 805-806).

Therefore, the present invention is related to a composition of adipose cell differentiation inhibition containing compound as shown in formula I:

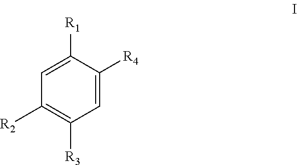

wherein R₁, R₃, or R₄ is C₁₋₆ alkoxyl group and R₂ is C₂₋₆ alkenyl group. In preferred embodiment, the compound of formula I is β-asarone with following chemical formula:

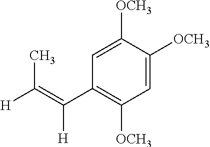

In preferred embodiment, the effective amount of formula I compound is at concentration of 0.015-0.25 mM, the most preferred concentration is 0.25 mM.

One mechanism of formula I compound on adipose cell differentiation inhibition is by inhibiting adipose cell differentiation transcription factors (such as PPARγ or C/EBPα mRNA or protein expression.

In preferred embodiment, above composition may contain other medically acceptable carrier, such as methanol or ethanol.

In the present invention, β-asarone is extracted from Acorus essential oil.

In the present invention, Acorus is an Acorus sp. plant, such as: Acorus calamus, Acorus tatarinowii, or Acorus gramineus, and the preferred one is Acorus calamus.

The present invention is also related to a composition of adipose cell differentiation inhibition containing effective amount of Acorus essential oil. In preferred embodiment, Acorus essential oil is at concentration of 0.015-480 mM, and the preferred concentration is 480 mM.

The present invention is also related to a composition of osteroporosis treatment containing compound of formula I:

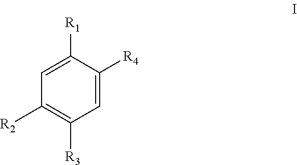

wherein R₁, R₃, or R₄ is C₁₋₆ alkoxyl group and R₂ is C₂₋₆ alkenyl group. In preferred embodiment, the compound of formula I is β-asarone with following chemical formula:

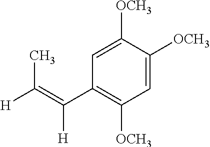

In preferred embodiment, the effective amount of formula I compound is at concentration of 0.015-0.25 mM, and the most preferred concentration is 0.25 mM.

One mechanism of formula I compound as a treatment for osteroporosis is by inhibiting PPARγ mRNA or protein expression. This inhibition enables embryo stem cell differentiate into osteoblast spontaneously.

In preferred embodiment, above composition for osteroporosis treatment may contain other medically acceptable carrier, such as methanol or ethanol.

In the present invention, β-asarone of the osteroporosis treatment composition is extracted from Acorus essential oil. Acorus is from Acorus sp. plant, such as Acorus calamus, Acorus tatarinowii, or Acorus gramineus, and the preferred one is Acorus calamus.

The present invention is also related to a composition for osteroporosis treatment containing effective amount of Acorus essential oil. In preferred embodiment, the concentration of Acorus essential oil is 0.015-480 mM, and the most preferred concentration is 480 mM. This composition may contain other medically acceptable carrier, such as methanol or ethanol.

### EXAMPLES

The present invention is using 3T3-L1 adipose cell differentiation level as screening criteria to examine the activity of 200 kinds of plant essential oil. Cell accumulated triglyceride is used as an indicator for selecting plant essential oil with activity. Further analyze its effect on adipose accumulation. Selected essential oil went through separation and purification process to identify its active ingredient. Its effect and molecular mechanism on adipose cell differentiation inhibition are also studied.

### Example 1

### 3T3-L1 Adipose Cell Differentiation Assay

In the present invention, 3T3-L1 fibroblast cell is purchased from Bioresource collection and research center. Medium for 3T3-L1 fibroblast cell culture contains 10% (v/v) FBS, Dulbecco's modified Eagle's high-glucose medium containing 1% (v/v) penicillin (10000 U/ml) and streptomycin (10 mg/ml). Cell culture is performed in 37 C, 10% CO2 incubators. Subculture is performed at 80-90% cell confluence. After cell reaches 100% confluence, differentiation solution is used for differentiation. The complete differentiation takes 9 days. Differentiation solution contains 0.5 mM IBMX, 10 μg/ml insulin, 0.5 μM dexamethasone in 10% FBS DMEM. Two or three days after 3T3-L1 fibroblast cell reaching 100% confluence (day 0), the original 10% FBS DMEM is removed and differentiation solution is added. Replace cell culture medium at day 3 and day 6 with 10% FBS DMEM containing 10 μg/ml insulin. Harvest cells at day 9 of differentiation for cell morphology observation (Juan, C. C.; Chang, C. L.; Lai, Y. H.; Ho, L. T., Endothelin-1 induces lipolysis in 3T3-L1 adipocytes. Am. J. Physiol. Endocrinol. Metab. 2005, 288, E1146-52).

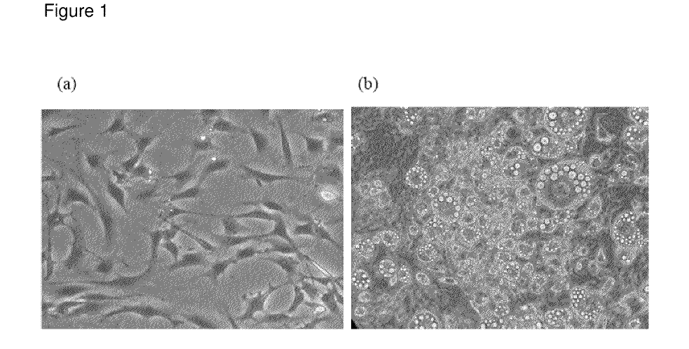

The result shows that 3T3-L1 pre-adipose cell can transform from spindle-shaped fibroblast to round-shaped cell. Oil drop is obvious to see in the cell (FIG. 1).

### Example 2

### Screen of Essential Oil Active Component on Adipose Cell Differentiation Inhibition

Mature 3T3-L1 adipose cell stores energy in the form of triglyceride of cell's oil drop. In order to study if plant essential oil is able to inhibit lipid accumulation in 3T3-L1 pre-adipose cell, we use cellular accumulated triglyceride content as an indicator of adipose cell differentiation. The examination method is as follows. Remove 3T3-L1 cell culture medium, wash cell with PBS for three times. Add protein lysis buffer (50 mM Tris-HCl, 150 mM NaCl, 0.5% sodium deoxycholate, 1% NP-40, 0.1% SDS) containing 10-fold diluted protease inhibitor into each well. Transfer mixture of scraped cells and buffer into microcentrifuge tube. Take 5 μl of homogenized cell mixture and 250 μl of reagent for reaction. The reaction is complete at room temperature in 1 hour, and then detects OD550 nm. Use OD value to calculate triglyceride concentration from a Merck multi-system calibrator generated standard curve. Determine triglyceride and protein concentration in each well, and the division of these two values (mg TG/mg protein) represents triglyceride content per unit protein in the cell.

Because essential oil contains more hydrophobic components, it is not easy to dissolve essential oil into medium. Therefore, amphipathic DMSO is used to assist essential oil into medium. The present invention used 200 different herbal essential oils from various plants. Add 100 μg/ml essential oil into 3T3-L1 pre-adipose cell for differentiation test, and replenish essential oil in culture medium to keep it steady during normal differentiation procedure. After completion of differentiation (day 9), triglyceride content analysis reveals that Acorus calamus essential oil has significant inhibition effect on adipose cell differentiation relative to its control (supplement with 0.1% DMSO). We then used Acorus calamus essential oil at various concentrations to examine 3T3-L1 pre-adipose cell differentiation again.

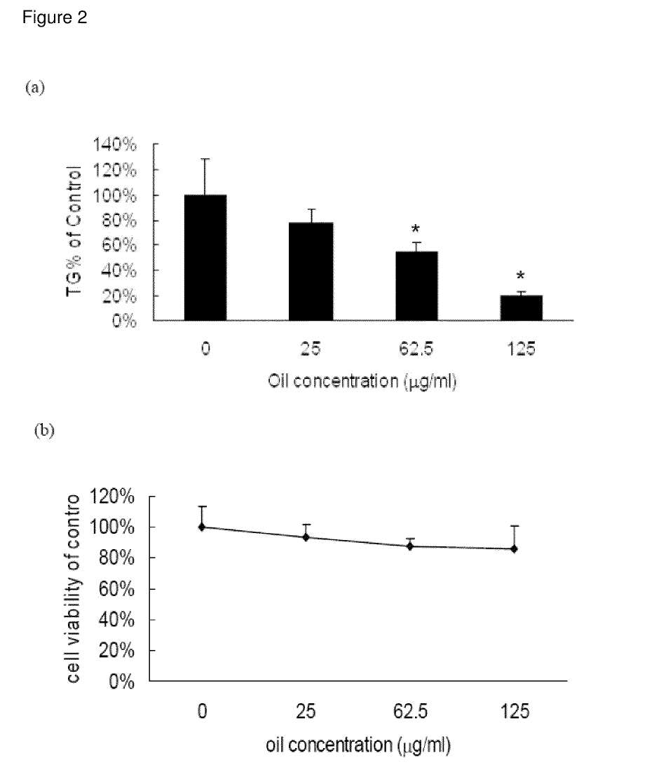

Dilute Acorus essential oil (density 1.10 g/ml) to 25, 62.5, and 125 mg/ml with DMSO. Until cell growth reaches post confluence (day 0), diluted Acorus essential oil solution is added into differentiation solution in 1:1000 dilution into cell (final concentration is 25, 62.5, and 125 μg/ml). While replacing growth medium containing insulin at day 3 and day 6, replenish each concentration of Acorus essential oil in culture medium as well as the ones adding 0.1% DMSO as controls. The analysis at day 9 discovered that 25 μg/ml Acorus essential oil reduces triglyceride content per cell unit protein to 77% relative to its control. Acorus essential oil at 62.5 and 125 μg/ml essential oil reduces it to 55% and 20% relative to its control (FIG. 2a). Besides, MTT assay for evaluating various concentrations of Acorus essential oil on cell viability effect reveals that even Acorus essential oil reaches its highest level of 125 μg/ml, cell viability is still maintained at 86% relative to normal differentiated control (FIG. 2b).

Acorus essential oil inhibition effect on cell differentiation can be visualized under microscope by oil red staining. It is performed as follows. Dissolve 0.03 g of Oil red O in 10 ml of isopropylalcohol as stock solution. Mix 2.4 ml of stock solution and 1.6 ml of PBS and filter it before staining. Remove medium from cells, add appropriate amount of filtered staining solution for 15 minutes, wash cell three times with PBS for 5 minutes each, and then add 4% paraformaldehyde for 15 minutes to fix cells, repeat wash step by PBS. Add 0.1% Triton X-100 for 30 seconds, and repeat wash step by PBS. Add appropriate amount of hematoxylin for 30 seconds followed by one last wash step by PBS. Observe the image under a microscope and record the image.

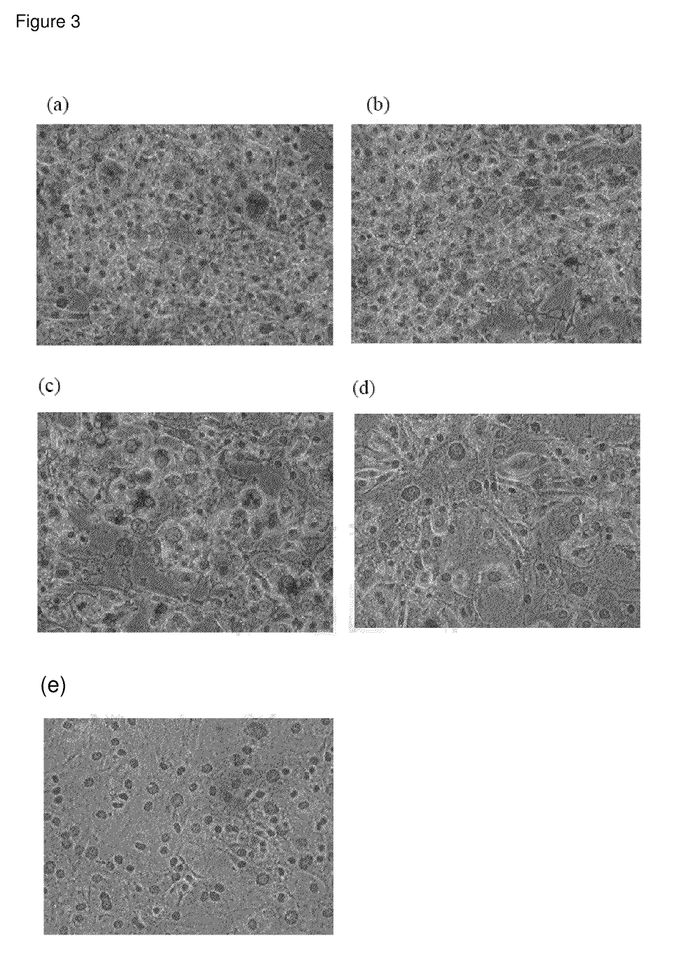

As shown in oil red staining result, elevated Acorus essential oil leads to less red oil in the cell (FIG. 3).

### Example 3

### Major Components Analysis of Acorus Essential Oil

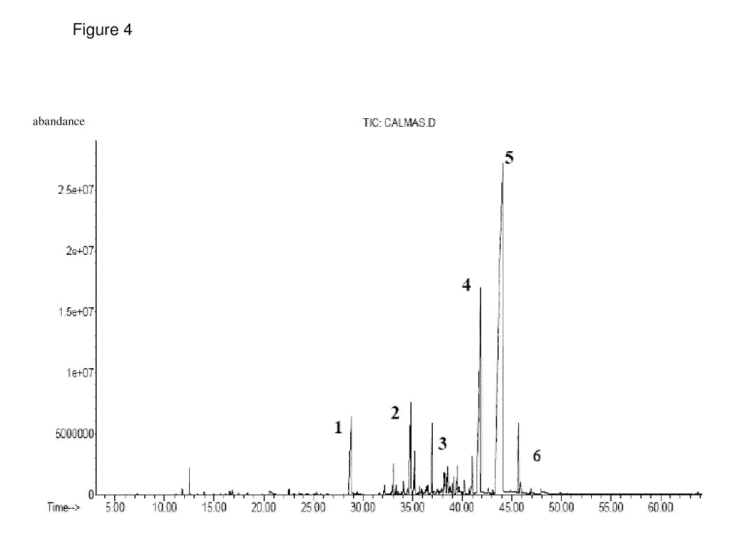

Dilute Acorus essential oil 10 fold with isopropylalcohol. At 250 C, use Agilent 7683 series autosampler injecting 1 μl of diluted essential oil sample into Gas Chromatography (Agilent 6890N). Capillary tube HP-5MS is 30 m×0.25 mm, and film thickness is 0.25 mm. Helium is used as mobile phase (1 mL/min) based on following conditions: starting temperature is 40 C/5 min, temperature heating rate is 3 C/min until 180 C. The second temperature ramp is at 6 C/min heating rate from 180 C to 200 C. The third temperature ramp is at 8 C/min heating rate from 200 C to 250 C, and temperature is kept at 250 C for 3 minutes. HP mass spectrometer 5973 is used for mass analysis, and detector temperature setting is 285 C. After comparing the resulting mass spectrometry (FIG. 4) with LC/MS library (Wiley275.L), it reveals that the major components in Acorus essential oil are β-asarone (56.67% of whole essential oil), euasarone, (17.39% of whole essential oil), cinnamaldehyde (4.65% of whole essential oil), methyl eugenol (3.79% of whole essential oil), trans-methylisoeugenol (2.46% of whole essential oil), and α-asarone (2.32% of whole essential oil) (Table 1).

TABLE 1
List of Acorus essential oil major components

| Peak No. | RT | Area Pct | Library/ID | CAS |
|---|---|---|---|---|
| 1 | 28.82 | 4.65 | cinnamaldehyde | 000104-55-2 |
| 2 | 34.81 | 3.79 | methyl eugenol | 000093-15-2 |
| 3 | 37.0 | 2.46 | cis-methyl isoeugenol | 006380-24-1 |
| 4 | 41.88 | 17.39 | euasarone | 005353-15-1 |
| 5 | 44.10 | 56.67 | beta-asarone | 005273-86-9 |
| 6 | 45.68 | 2.32 | alpha-asarone | 005273-86-9 |

### Example 4

### Silica Gel Chromatography and Cell Differentiation Inhibition Assay of Each Fraction

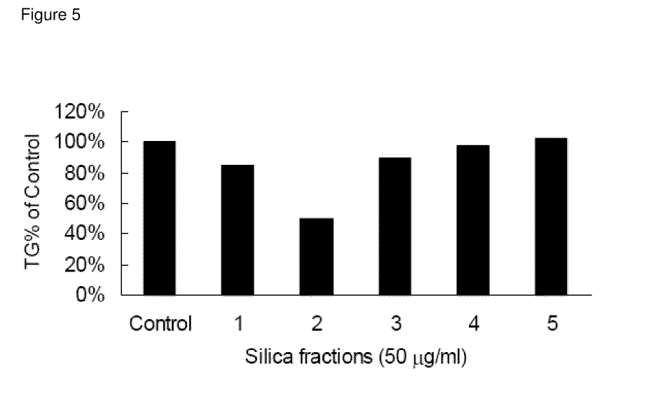

Ten ml of Acorus essential oil dissolved in hexane (5 ml essential oil and 5 ml hexane) is loaded into prepacked silica gel glass column and eluted by hydrophobicity (Table 2). Essential oil can be separated into 5 portions based on each component's hydrophobicity. Remove elution solvent by lypholizer, dissolve each component in 2 ml DMSO or methanol, and store it in glass vial. 3T3-L1 is used as a cell model, we add various fractions in DMSO or methanol at concentration of 50 μg/ml into cell differentiation medium and growth medium. At day 9 of complete differentiation, analyze triglyceride content per cell unit protein. The result shows that triglyceride content of cells with 50 μg/ml essential oil first fraction supplement is 85% relative to its control (0.01% DMSO). The 2, 3, 4, and 5 fraction contains 50%, 90%, 98%, and 103% of triglyceride content comparing to its control (FIG. 5). The result shows that the second fraction from Acorus essential oil has the maximal activity on cell differentiation inhibition. Therefore, the second fraction has been chosen for further analysis.

TABLE 2
Mobile phase composition of liquid chromatography

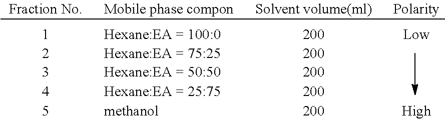

Mix hexane and ethyl acetate in various proportions. The fifth fraction is eluted by methanol.

### Example 5

### HPLC Analysis and Major Component Identification

After confirming the major component of Acorus essential oil is in the second fraction, HPLC is used to further separate this fraction. The composition of its mobile phase is shown in Table 3. Inject 10 μl of silica gel chromatography column purified fraction into HPLC (Agilent HPLC 1100) with silica packed column (250×4.6 mm×5 μm). The mobile phase is mixed by hexane and EA in various proportions (90/10, 65/35, and 20/80) to generate concentration gradient, flow rate is 0.03 ml/min, analysis is finished in 30 min, and OD210 nm is used to analyze result.

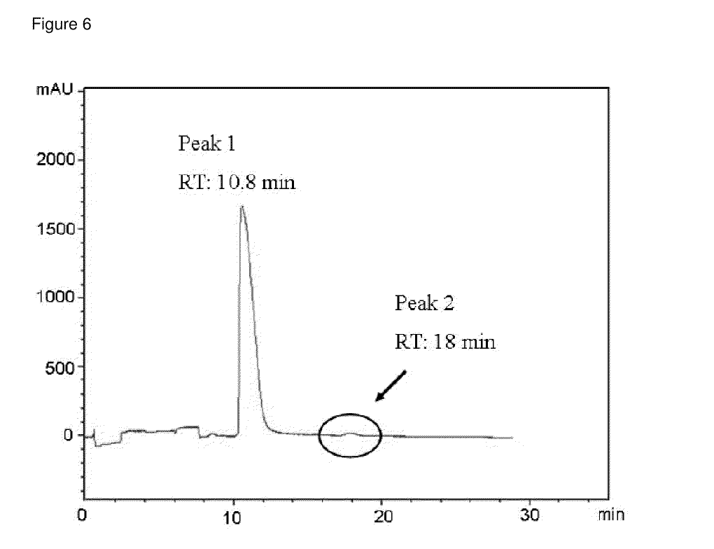

The result shows a peak at 10.8 min retention time, and another smaller peak is also present at 18 min (FIG. 6). Collect these two peaks separately in microcentrifuge tubes from 10 samples. Analyze these two samples by GC-MS and compare them to MS library. The result shows that peak 1 of 10.8 min retention time is β-asarone and peak 2 of 18 min retention time is α-asarone.

TABLE 3
The composition of HPLC mobile phase

| Time (min) | Hexane | Ethyl acetate | Flow rate (ml/min) |
|---|---|---|---|
| 0 | 90 | 10 | 0.03 |
| 5 | 65 | 35 | 0.03 |
| 30 | 20 | 80 | 0.03 |

Mix hexane and ethyl acetate in various proportions. Flow rate is 3 ml/min.

### Example 6

### Analysis of α-Asarone and β-Asarone Inhibition Effect on Adipose Cell Differentiation

After identifying the second fraction of Acorus essential oil as α-asarone and β-asarone, we further examine the effect of these two compounds on adipose cell differentiation inhibition. We dissolve α-asarone and β-asarone in DMSO to make concentration of 0.015, 0.03, 0.06, 0.13, and 0.25 M solution. Add compounds in 1:1000 dilution into 3T3-L1 cell differentiation medium or growth medium according to differentiation process. Cell supplemented with 0.1% DMSO is used as a normal differentiation control. Analyze triglyceride content per cell unit protein at day 9 of complete differentiation.

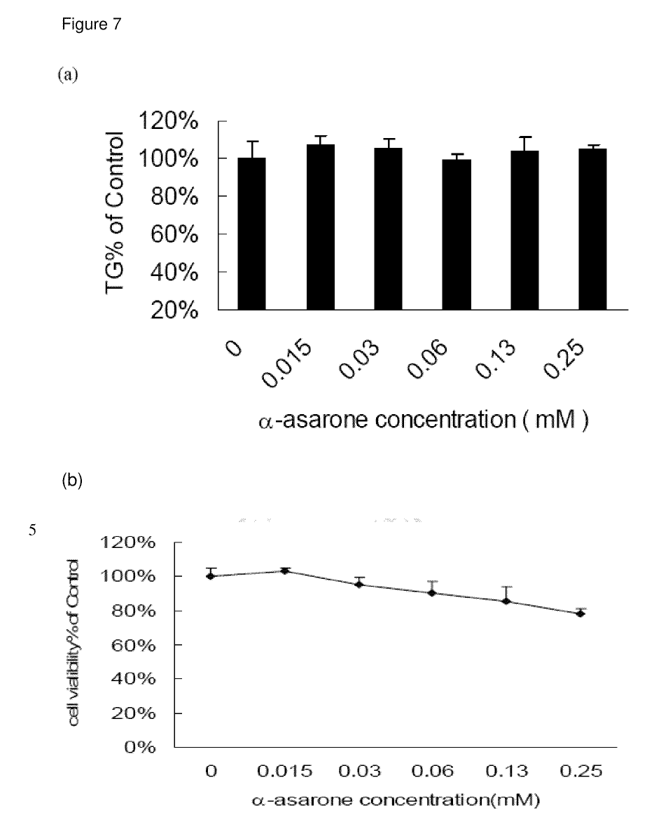

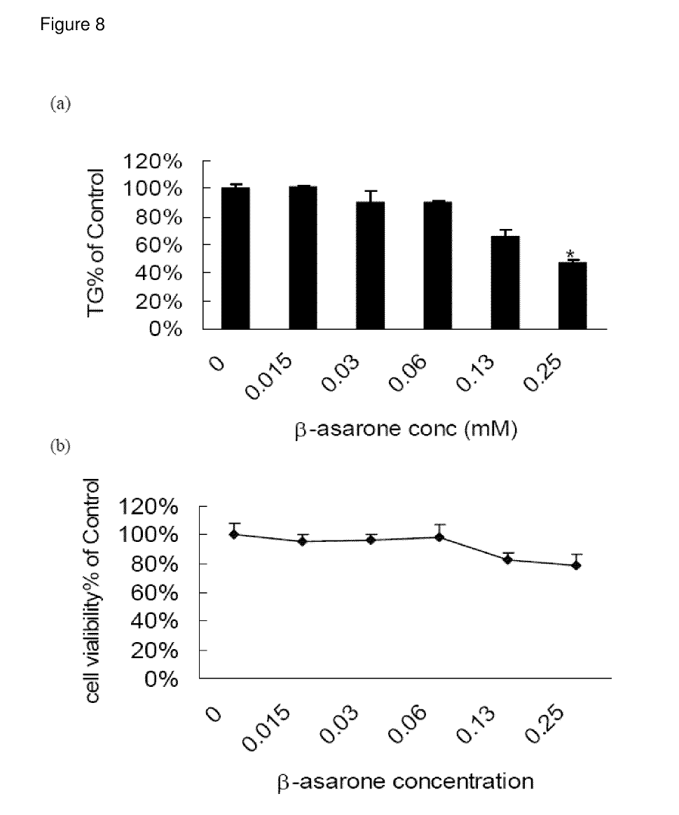

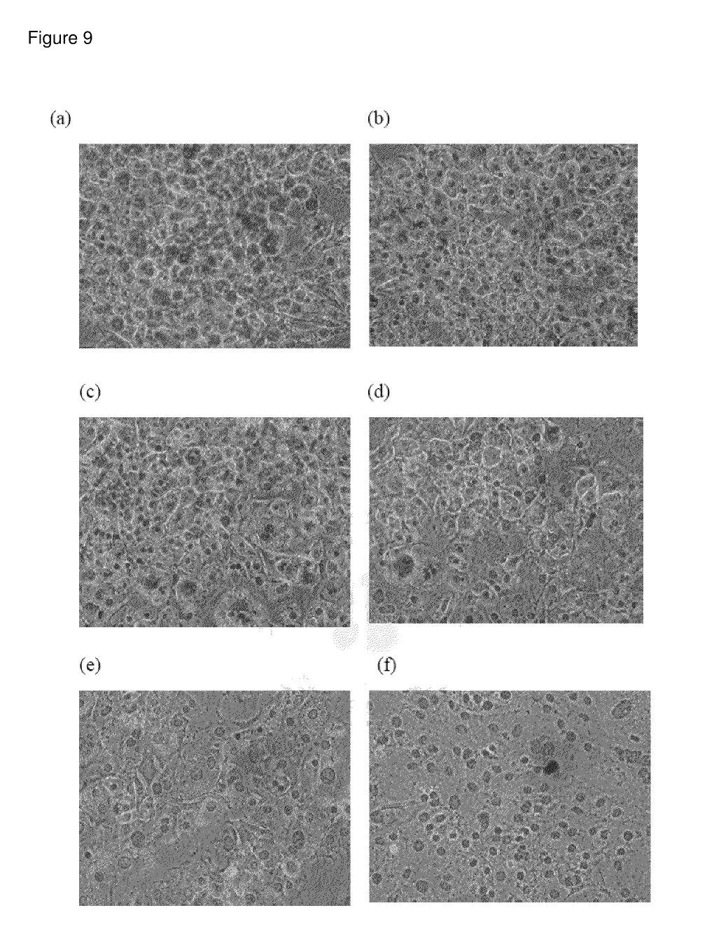

The result shows that triglyceride content of α-asarone treated 3T3-L1 cell (final concentration of 0.015, 0.03, 0.06, 0.13, and 0.25 mM) is 107%, 106%, 99%, 104%, and 105% relative to its normal differentiation control (FIG. 7a) without significant variations. Cell viability analysis shows that cell viability rate of maximal amount of α-asarone (0.25 mM) treated cell is 78% relative to the normal differentiated control (FIG. 7b). Triglyceride content of β-asarone treated 3T3L-1 cell (final concentration of 0.015, 0.03, 0.06, 0.13, and 0.25 mM) is 101%, 90%, 91%, 66%, and 47% relative to its normal differentiated control (FIG. 8a). With elevated amount of β-asarone, cellular triglyceride content is decreasing. Staining β-asarone treated 3T3-L1 cell with oil red, it is observed that red oil covered area is decreasing as β-asarone arising (FIG. 9). It shows that β-asarone can effectively inhibit oil accumulation in 3T3-L1 cell. Cell viability analysis shows that treated cell viability of maximal amount of β-asarone (0.25 mM) treated cell is 79% relative to its normal differentiated control (FIG. 8b).

### Example 7

### Molecular Mechanism of β-Asarone Inhibition Effect on Adipose Cell Differentiation

After identifying β-asarone as the active component in Acorus essential oil second fraction for inhibiting adipose cell differentiation, we select two adipose cell differentiation related transcription factor marker PPARγ and C/EBPα to test their expression of β-asarone treated 3T3-L1 cell during differentiation.

mRNA Level Expression of PPARγ and C/EBPα

RNA extraction: Remove 3T3-L1 cell medium directly from 10 cm petri dish, add 1 ml of Trizol reagent, and scrape off cells. Transfer cell and reagent mixture into a microcentrifuge tube. Add 200 μl chloroform in each tube and vortex for 15 seconds until it is clouded. Leave it on ice for 2-3 minutes at 4 C, spin it at 12000 rpm for 20 minutes. Transfer supernatant to another microcentrifuge tube, add equal amount of isopropylalcohol, mix and leave it on ice for 10 minutes. Spin it at 12000 rpm for 10 minutes at 4 C. After centrifugation, remove supernatant and save RNA pellet in the bottom. Add 75% cold ethanol, spin it at 7500 rpm for 10 minutes at 4 C. Remove ethanol after centrifugation and air dry the pellet until the edge is clear. Add appropriate amount of DEPC H₂O to dissolve RNA pellet, and determine RNA concentration by OD260/OD280 nm with a spectrophotometer.

RT-PCR: Take 2 μg of RNA for reverse transcription reaction. Mix RNA, DEPC H₂O and 0.25 μg random primer to make total volume of 12 μl. Keep it at 70 C for 10 minutes, and then leave it on ice. Add 4 μl of 5× first-strand buffer, 2 μl of 0.1 M dithiothreitol, 1 μl of 10 mM dNTP and 1 μl of SuperScript™ II reverse transcriptase 200 U/μl. Keep it at 42 C for 1.5 hours, and then heat it at 70 C for 15 minutes to stop reverse transcription. Take 1 μl of cDNA from reverse transcription, mix with 5 μl of 10×PCR buffer, 1 μl of 10 mM dNTP, 2 μl of primer, 40.5 μl of sterile water, and 0.5 μl of Taq DNA polymerase (1 U/μl) for PCR reaction. Primers sequences are listed in Table 4. The SEQ ID NO: 1 to SEQ ID NO: 6 is listed from the top to bottom accordingly. PCR program is listed as follows: 95 C/5 min; 25˜30 cycles of 95 C/1 min, 55 C/1 min, 72 C/1 min; 72 C/10 min, and stop reaction at 4 C.

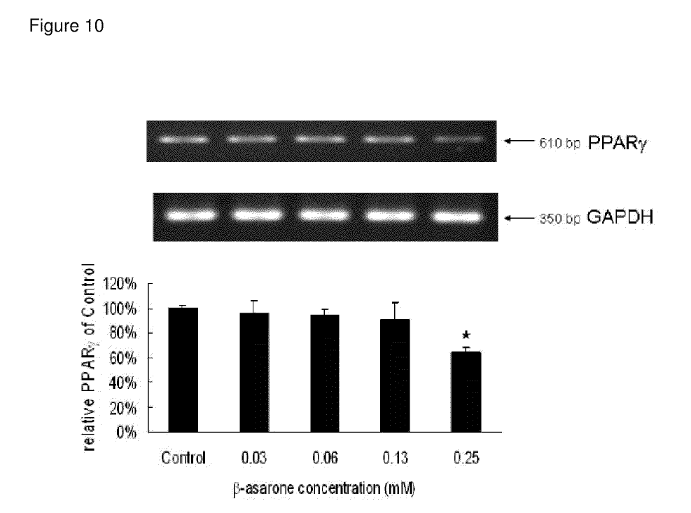

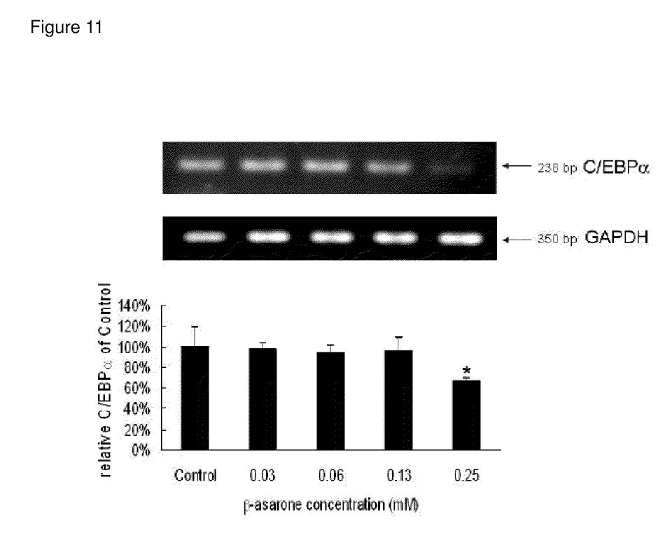

Because PPARγ and C/EBPα are early marker genes during adipose cell differentiation, these genes have been analyzed from cells harvested after 3 days of β-asarone addition. By RT-PCR analysis, β-asarone at concentration of 0.03, 0.06, and 0.13 mM cause slight decrease of PPARγ expression of 96%, 94%, and 90% relative to its normal differentiated control with no significant variation. When β-asarone reaches 0.25 mM, expression of PPARγ reduces to 64% relative to its control (FIG. 10). In terms of another differentiation marker gene C/EBPα, β-asarone at concentration of 0.03, 0.06, and 0.13 mM slightly decreases C/EBPα expression to 99%, 94%, and 96% relative to its normal differentiated control with no statistical difference. However, if β-asarone reaches 0.25 mM, the expression of C/EBPα decreases to 66% relative to its control (FIG. 11).

TABLE 4
PCR primer sequence

| Gene name | Left/right primer | size |
|---|---|---|
| PPARγ | ACCACTCGCATTCCTTTGAC | 610 bp |
| | TCAGCGGGAAGGACTTTATG | |
| C/EBRα | AGGTGCTGGAGTTGACCAGT | 238 bp |
| | CAGCCTAGAGATCCAGCGAC | |
| GAPDH | GTGTTCCTACCCCCAATGTG | 350 bp |
| | CTTGCTCAGTGTCCTTGCTG | |

### Protein Level Expression of PPARγ and C/EBPα

Western blot analysis: Prepare 12% SDS-PAGE as follows. Recipe of separating gel contains 2.5 ml of 1.5 M Tris-HCl pH 8.8, 100 μl of 10% SDS, 3 ml of acrylamide/bis (37.5:1; 40%), 4.35 ml RO water, 50 μl of 10% ammonium persulfate, and 5 μl of TEMED. Recipe of stacking gel contains 1.25 ml of 0.5 M Tris-HCl pH 6.8, 50 μl of 10% SDS, 0.065 ml of acrylamide/bis (37.5:1; 40%), 3.05 ml of RO water, 25 μl of APS and 5 μl of TEMED. Load same amount of protein in each well, and electrophoresis is performed under 100V for 2 hours. Prepare transfer buffer (10 mM CAPS-NaOH, 10% methanol, pH 11.0), and use methanol to rinse PVDF transfer membrane. After electrophoresis is complete, remove SDS-PAGE gel, cover the gel and PVDF membrane with transfer buffer rinsed 3 MM paper. Put it into transfer tank full of transfer buffer, electrophoresis under 500 mA for 1 hour. Take transfer membrane out, block it with 5% skim milk in TBST (20 mM Tris, 137 mM NaCl, 0.01% Tween-20), and wash it with TBST briefly. Add primary antibody for 2-hour reaction, wash membrane with TBST for three times, 15 minutes each. Add appropriate amount of secondary antibody for 1 hour, wash membrane with TBST for three times, 15 minutes each. In the end, evenly cover ECL on the transfer membrane, place membrane in the film cassette, and expose it to X-ray film. Develop the film for analysis.

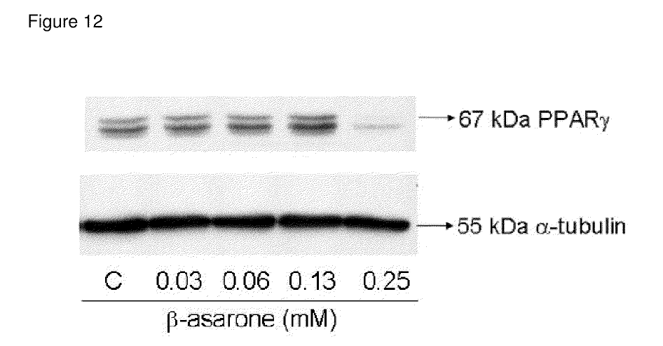

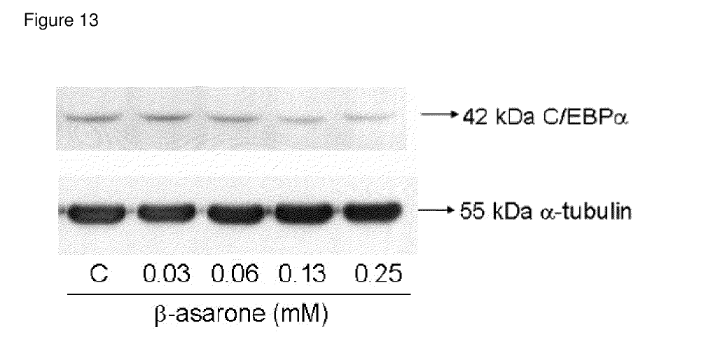

Analyze cells of β-asarone supplement for 3 days by western analysis. The expression of PPARγ from cells treated with β-asarone at concentration of 0.03, 0.06, and 0.13 mM is 97%, 117%, and 111% relative to its normal differentiated control with no significant difference. When β-asarone reaches 0.25 mM, the expression of PPARγ reduces to 45% relative to its control (FIG. 12). In terms of another differentiation marker gene C/EBPα, the expression of C/EBPα treated with β-asarone at concentrations of 0.03 and 0.06 mM is 113% and 77% relative to its control with no statistical difference. However, when β-asarone reaches 0.13 mM and 0.25 mM, the expression of C/EBPα reduces to 43% and 33% relative to its control (FIG. 13).

What is claimed is:

1. A method for inhibiting adipose cell differentiation comprising administering to a subject in need thereof an effective amount of compound in formula I: 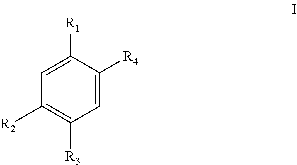
wherein R₁, R₃ or R₄ is C₁₋₆ alkoxyl group and R₂ is C₂₋₆ alkenyl group.

2. The method as claimed in claim 1, wherein said compound in formula I is β-asarone exhibiting following chemical formula:

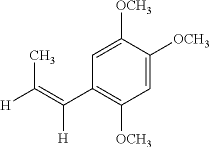

3. The method as claimed in claim 1, wherein said compound concentration of formula I is 0.015-0.25 mM.

4. The method as claimed in claim 3, wherein said compound concentration of formula I is 0.25 mM.

5. The method as claimed in claim 1, wherein said compound of formula I can inhibit transcription factor expression of adipose cell differentiation.

6. The method as claimed in claim 5, wherein said transcription factor is PPARγ.

7. The method as claimed in claim 5, wherein said transcription factor is C/EBPα.

8. The method as claimed in claim 1, wherein the compound further comprises other medically acceptable carrier.

9. The method as claimed in claim 8, wherein said carrier is methanol or ethanol.

10. The method as claimed in claim 2, wherein said β-asarone is extracted from Acorus essential oil.

11. The method as claimed in claim 10, wherein said Acorus belongs to Acorus sp.

12. The method as claimed in claim 11, wherein said Acorus comprises Acorus calamus, Acorus tatarinowii or Acorus gramineus.

13. The method as claimed in claim 12, wherein said Acorus is Acorus calamus.

14. A method for inhibiting adipose cell differentiation comprising administering to a subject in need thereof an effective amount of Acorus essential oil.

15. The method as claimed in claim 14, wherein said Acorus belongs to Acorus sp.

16. The method as claimed in claim 15, wherein said Acorus comprises Acorus calamus, Acorus tatarinowii or Acorus gramineus.

17. The method as claimed in claim 16, wherein said Acorus is Acorus calamus.

18. The method as claimed in claim 14, wherein said effective amount of Acorus essential oil is between 0.015-480 mM.

19. The method as claimed in claim 18, wherein said effective amount of Acorus essential oil is 480 mM.

20. The method as claimed in claim 14, wherein the Acorus essential oil comprises other medically acceptable carrier.

21. The method as claimed in claim 20, wherein said carrier is methanol or ethanol.

22. A method for treating osteoporosis comprising administering to a subject in need thereof an effective amount of compound of formula I:

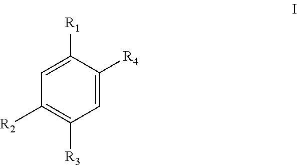

23. The method as claimed in claim 22, wherein said compound of formula I is β-asarone with following chemical formula:

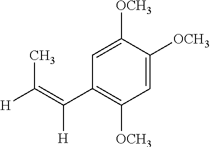

24. The method as claimed in claim 22, wherein concentration of said compound I is between 0.015-0.25 mM.

25. The method as claimed in claim 24, wherein concentration of said compound is 0.25 mM.

26. The method as claimed in claim 22, wherein said compound of formula I can inhibit PPARγ expression.

27. The method as claimed in claim 22, wherein said compound of formula I comprises other medically acceptable carrier.

28. The method as claimed in claim 27, wherein said carrier is methanol or ethanol.

29. The method as claimed in claim 23, wherein said β-asarone is extracted from Acorus essential oil.

30. The method as claimed in claim 29, wherein said Acorus belongs to Acorus sp.

31. The method as claimed in claim 30, wherein said Acorus comprises Acorus calamus, Acorus tatarinowii or Acorus gramineus.

32. The method as claimed in claim 31, wherein said Acorus is Acorus calamus.

33. A method for treating osteoporosis comprising administering to a subject in need thereof an effective amount of Acorus essential oil.

34. The method as claimed in claim 33, wherein said Acorus belongs to Acorus sp.

35. The method as claimed in claim 34, wherein said Acorus comprises Acorus calamus, Acorus tatarinowii or Acorus gramineus.

36. The method as claimed in claim 35, wherein said Acorus is Acorus calamus.

37. The method as claimed in claim 33, wherein said effective amount of Acorus essential oil is 0.015-480 mM.

38. The method as claimed in claim 37, wherein said effective amount of Acorus essential oil is 480 mM.

39. The method as claimed in claim 33, wherein Acorus essential oil comprises other medically acceptable carrier.

40. The method as claimed in claim 39, wherein said carrier is methanol or ethanol.
